(19) United States
(12) Patent Application Publication
(10) Pub. No.: US 2002/0147390 A1
(43) Pub. Date: Oct. 10, 2002

(54) Methods and apparatus for acquiring and using bedside medical data

(76) Inventors: John Emmanuel M.D. Markis, Waban, MA (US); E. John Groener, Acton, MA (US); Richard H. Baker, (US); William S. Oncay, Whitinsville, MA (US); Anthony G. P. Marini, Clinton, MA (US)

Correspondence Address: CHARLES G. CALL, 68 HORSE POND ROAD, WEST YARMOUTH, MA, 02673-2516, US

(21) Appl. No.: 10/034,346

(22) Filed: Dec. 20, 2001

Related U.S. Application Data
(60) Provisional application No. 60/257,088, filed on Dec. 20, 2000.

(51) Int. Cl. A61B 5/00

(52) U.S. Cl. 600/301; 235/375

(57) ABSTRACT

A system for gathering, displaying and analyzing vital sign data hospital patient. A handheld computer including a touchscreen and a barcode scanner is linked via a WLAN network to a database server which stores patient data at a central location. The handheld computer includes a browser program for displaying HTML data entry forms transmitted from the database server which the nurse/operator uses to supply vital signs data such as the patient's current temperature, blood pressure, pulse rate, and respiration rate. Further data entry forms allow the entry of body intake and output amounts and the patients weight. A barcode scanner integrated with the handheld computer may be used to read patient and operatior identification values, as well as medication identification values, from printed media. The touchscreen controls allow current vital sign values to be incremented or decremented. A conventional PC secured to the patients room may be used when appropriate to perform the bedside data collection and display functions performed by the handheld device.

### CROSS-REFERENCE TO RELATED APPLICATION

This application claims the benefit of the filing date of the copending U.S. Provisional Patent Application Serial No. 60/257,088 filed on Dec. 20, 2000.

### FIELD OF THE INVENTION

This invention relates to medical information systems and more particularly to methods and apparatus for acquiring bedside medical data.

### BACKGROUND AND SUMMARY OF THE INVENTION

Presently almost all patient medical information is available at multiple computer workstations on the floors of most hospitals in the United States. Information such as chest X-ray reports, daily blood test results and even the display of an ECG can be easily accessed with a few simple keystrokes in the nursing station away from the patient bedside. In addition, other information such as patient demographics, reference guides to patient care and e-mail are all easily available, not only at nursing stations but in physician offices and homes, and can be remotely accessed by establishing a dialup connection to the hospital patient data base. Internet access is also now widely used at all nursing stations and there is progress toward making the patient information database available with restrictions via the Internet.

Despite the computerization of virtually all patient information, there is data that is very important to patient care which still resides exclusively on hand-entered charts and forms at the patient's bedside. Vital sign information such as blood pressure, pulse, respirations, heart rate, percent blood oxygen saturation by oxymetry, weight, fluid intake and output of body fluids, tests for blood in stool, IV solutions, type, time and amount given are all charted on multiple pages by hand and attached to bedside clipboards and/or affixed to a bulletin board in the patient's room. In order to access this important data, which is also very closely related to the data that is already in the nursing station computer, one must examine the charted sheets at the bedside separately and then make clinical judgments at the nursing station in order to perform entries for the patient away from this charted information. The attending physician away from the hospital in his office can access the latest chest x-ray report, the latest blood tests, or lists of medications the patient is taking using his office computer, but there is no way to know the latest blood pressure, temperature or heart rate, or example, in response to some intervention that was performed or medicine given while the physician was at the hospital a few hours before or the subsequent response of the patient to the intervention. The hospital floor must be called to acquire this data from the nurse or the medical house staff, who may not remember accurately the needed information without going into the patient's room while on the phone to read the various charts. Often the nurse or medical house officer may not even know the most recent information about the patient since the information may have been charted by a nurse's aide or nurse assistant. This further complicates data retrieval.

Moreover, there is presently no means for accurately identifying the person who actually acquired the data and made the entry, since, as a matter of routine, noone initials or personalizes each piece of data. Thus, no individual responsibility may exist as to the accuracy of the data. In addition, the restricted means of access diminishes the attention and extent to which the data is monitored and used by the persons responsible for the patient's care. Often, the sum of the patient's oral or intravenous input is often not calculated except ad hoc, delaying and complicating the acquisition of data relating to a shift or 24-hour period. Realizing the present inaccuracies inherent in the system, the data may even be ignored by caregivers. Further, archiving and record keeping is made difficult by this system due to the need for filing and storing of multiple sheets of charted preprinted, one-of-a-kind forms.

No mechanism for recording the actual administration of medications, oral or intravenous blood products, treatments, response to treatments, or for the verification of the identity of patients and caregivers, is currently available at the patient bedside.

The handwritten entries may be difficult to interpret, particularly by persons other than the one making the entries. The conventional charts upon which the handwritten entries are made are typified by the handwritten bedside charts found in Part A of the Appendix. Although the printed forms allocate space for different kinds of information, these charts provide no mechanism for helping to insure that the entries made are complete, accurate, and in compliance with desired procedures.

### SUMMARY OF THE INVENTION

In a principle aspect, the present invention takes the form of methods and apparatus for automating the collection and use of bedside patient data.

In the preferred embodiment of the invention, a bedside data terminal which is linked to the hospital's medical information system incorporates a touch screen interface which both displays patient data and allows that data to be easily updated by simply touching the touch-screen interface. Bedside data is displayed in numerical format together with active touchscreen areas that allow the displayed numerals to be incremented upwardly or downwardly from current or nominal values. On request, the bedside data terminal can be used to display collected bedside data in historical chart form.

As used in this specification, the term “bedside data” refers to the data which, in the past, has been traditionally entered by hand on charts and forms at the hospital patient's bedside and includes vital sign information such as blood pressure, respiration and pulse rates, urine and other fluid outputs, and the amount and administration times of IV solutions of specified types as well as other fluid intakes. In addition, the collected bedside data may include oxygen saturation by oxymetry, the patient's weight, and tests for blood in stool. In addition, the bedside data acquired in accordance with the present invention includes the identification of the patient as well as the person who collects the data, and optionally permits spoken comments on the patient to be entered and stored, as noted below. All data that is entered is preferably automatically time-stamped, patient-stamped and nurse-stamped.

As further contemplated by the invention, the bedside data terminal may incorporate a microphone for recording comments on the patient's condition which are stored in the medical information system for local playback at the bedside terminal, or at any other location connected to the hospital's information system. To facilitate hands-free operation, and as alternative to manipulating the touch screen, spoken commands may be accepted via the microphone associated with the bedside unit to set bedside data variables.

The bedside data terminal may advantageously take the form of a handheld data collection device that is coupled by a wireless link to central database server than may be integrated with the existing hospital patient monitoring and medical information system so that data entered into the terminal becomes instantly available. As an alternative, the handheld device may simply accumulate the patient data in its memory and then transfer this data to the medical information system by a wired connection or by a media transfer.

The bedside data terminal may alternatively be implemented by a personal computer that remains in the patient's room. To prevent theft, the device is preferably permanently secured at a convenient location to a wall or to an object that cannot easily be removed without detection, such as a table or the patient's bed. While the use of a fixed terminal eliminates any requirement that the staff member carry a handheld unit; handheld units may be used to advantage to reduce costs by serving multiple bedside locations that cannot be easily equipped with fixed bedside terminals.

Since leaving the patient's room to enter vital signs data would degrade the accuracy of the data as well as consume valuable time, the bedside data acquisition device should be located near each patient's bedside for convenient use when bedside measurements and procedures are being performed. This can be achieved either by using the preferred touchscreen, hand-held computer or a fixed terminal which may be conveniently positioned near each patient's bedside.

The bedside data is preferably gathered using a terminal computing device which executes a browser program for displaying HTML pages generated by a database server which stores clinical data. The use of the open standard, browser-based data collection and delivery system permits a variety of bedside devices to be used, including standard, hand-held “personal digital assistant” devices which employ touch screens, such as those based on the Palm or Pocket PC operating systems which preferably incorporate or connect to bar-code scanners. In addition, standalone, room-mounted PCs that are preferably (but not necessarily) equipped with touch screens and bar-code scanners may be employed to collect and display bedside data.

The database server is preferably integrated with or connected to the existing hospital information system (HIS) and communicates with the bedside a wired connection of various types (a modem and telephone line, local area network Ethernet connection, or the like), or by a radio frequency network such as an IEEE 802.11 WLAN or by a “Bluetooth™” short range radio link.

The bedside unit includes means for accepting data identifying the person, such as a nurse or other caregiver, who is gathering the bedside data, as well as additional data identifying the patient. While the caregiver's identification number, and identification data carried on the patient's wristband, may be manually entered into the data collection device, the handheld data collection terminal may be advantageously provided with a bar-code scanner for accurately and automatically reading the care-giver's identification from a barcoded badge or the like, and reading the patient's identification from a barcoded wristband worn by the patient. The acquired patient identification data further acts as the primary key value used by the medical information system to associate the collected data with other data relating to that patient in the database. The barcode scanner may also be used to scan identification numbers from containers holding medication administered at the bedside.

The bedside data terminal preferably automatically prompts the user for the data to be entered in a predetermined sequence in accordance with a standard procedure, and further permits the user to enter data selected by the user. As soon as the data is entered, it is preferably transmitted to the hospital computer system from which it may be immediately accessed in a form that preferably resembles the handwritten data sheets traditionally found at the patient's bedside. By closely following the format of conventional bedside charts, doctors and nurses who use the computer-generated data readily understand its meaning and significance without special training. If desired, the data collected by the bedside terminal may be combined with the data from the medical information system for display or printing when desired.

In addition, the vital signs data collected by the bedside data terminal unit may be displayed in a graphical format on a scrollable screen display, permitting the viewer to scroll back in time to data from for example, several days before, to examine trends, to access averaged data and relate all of this information to that which is available at the same time at the same workstation terminal. The patient vital sign display at the computer terminal could also be annotated with dictated or keyboarded comments, such as patient appearance or behavior, or noting that a medication was begun or discontinued.

Because the data being entered is primarily numerical, it may entered using a touch screen numerical display coupled with touch areas that accept commands to increment or decrement a displayed numerical value. In addition, voice recognition techniques that may be used employed to permit the caregiver respond to visible or audio prompts with spoken data values, freeing the hands of the caregiver to perform related functions as the data is being acquired.

Order entry may be facilitated at the bedside from a source of pharmacy orders with access to drug availability, referenced to patient allergy history, drug intolerance as a consequence of current status (e. g. treatment with anticoagulants), other tests and procedures, access to data and the times of scheduled procedures and interventions.

As contemplated by the present invention, the gathering of bedside data is automated for convenience, efficiency and enhanced safety; it is more accurate, more complete, and more accountable, while at the same time this data readily combined with other patient data in the hospital's medical information systems for easy access to multiple on-site and off-site users, while reducing the problems of archiving and storing the data for later use when needed.

These and other features and advantages of the present invention may be more readily understood by considering the following detailed description. In the course of this description, reference will be made to the attached drawings.

### BRIEF DESCRIPTION OF THE DRAWINGS

FIG. 1 illustrates the basic components used to implement a bedside monitoring system that utilizes the invention;

FIG. 2 shows an illustrative data entry screen display/edit screen displayed by the bedside data entry, editing and display terminal contemplated by the invention;

FIG. 3 illustrates a screen display used to confirm the accuracy of the data recorded for a patient at the conclusion of a data entry session;

FIG. 4 depicts an illustrative trend display screen display; and

FIG. 5 illustrates the input/output data recorded for a patient displayed in tabular form.

### DETAILED DESCRIPTION

Hardware Architecture

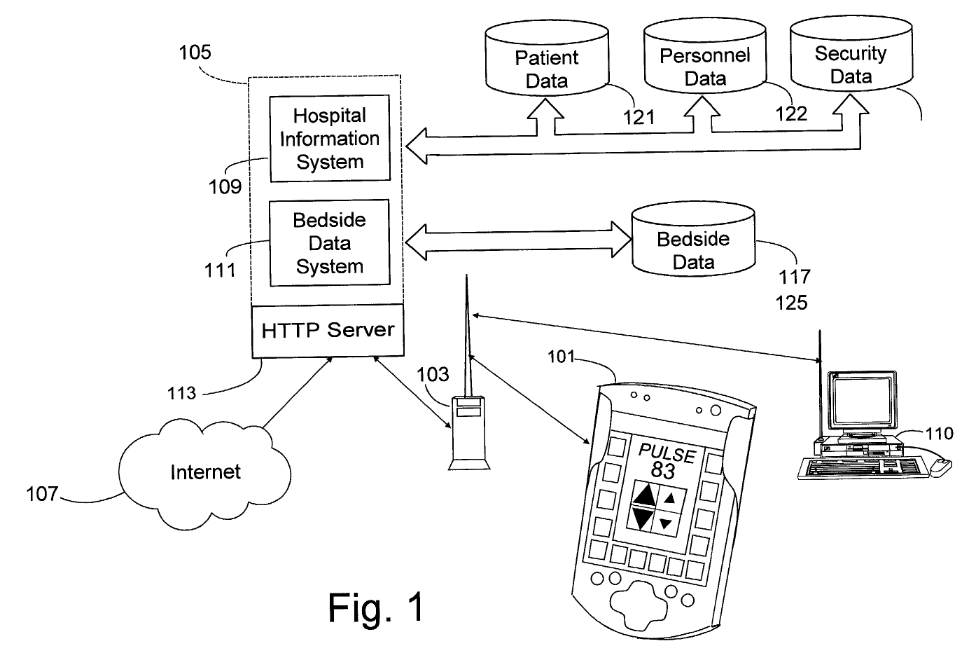

As illustrated in FIG. 1, the invention may be implemented by one or more handheld web browser-based bedside units 101 and/or fixed personal computer bedside units 110 linked via a radio frequency network base station 103 to a database server indicated generally at 105.

The bedside unit(s) 101 are preferably portable, hand-held computing devices that incorporate a touch screen for display and data entry, as well as a barcode scanner capable of reading printed barcode values that identify patients, hospital personnel, and medications. The handheld bedside unit may be implemented by a personal data assistant (PDA) which employs the Palm or Windows CE operating system and which executes a conventional browser program for displaying HTML “web pages.” For example, the handheld computer may consist of an iPAQ™ Pocket PC H3600 or H3100 Series handheld computer manufactured by Compaq Corporation combined with the SPS 3000 barcode scanner expansion pack manufactured by Symbol Technology which supplies built in WLAN connectivity through an integrated Spectrum24® 802.11b radio and antenna which communicates with a 802.1lb base station 103 which is in turn connected to the database server 105 via a wired or wireless connection. The IEEE 802.11 wireless local area network (WLAN) is described in detail in “The IEEE 802.11 Handbook: A Designer's Companion” By Bob O'Hara & Al Petrick, ISBN 0-7381-1855-9 (1999).

The database server 105 web server includes a conventional HTTP server 113 for exchanging information using the hospital's WLAN network with the browser programs executed by the bedside units, and with remote computing devices (not shown) connected via the Internet 107. The database server, which may comprise a single computer or a plurality of cooperating computers, provides services from both a conventional hospital information system (HIS) 109 and the bedside data system 111 which implements the present invention.

The browser-based nature of the bedside data system allows its services to delivered to the bedside using a wide variety of industry-standard devices. In addition to the handheld computing unit described above, a standalone, room-mounted industrial PC as illustrated at 110, provided with a touchscreen and barcode scanner for data-entry ease, may be employed. The handheld and conventional PC's use the same wireless networking connection to the database server and can display the collection and analysis applications in a web browser. All data collection and analysis functions are provided via the web browser.

The database server machine(s) 105 executes the HTTP web server application seen at 113 for delivering HTML “web pages” which provide data collection screens and/or data analysis displays to the bedside and which connect to a database “back end” bedside data system program 109 for storing the clinical bedside data at 117. The bedside data system 111 preferably communicates with a conventional hospital information system (HIS) 109 which provides patient identification information stored at 121, personnel information at 123 identifying hospital staff members, and security data (usernames, passwords and encryption keys) at 125 which are used to prevent unauthorized access to information stored in both the HIS and bedside data systems.

In a typical installation, the bedside data system contemplated by the invention will employ some number of bedside data collection stations 101 and 110, depending on the number of beds in the healthcare facility, and at least one database server computer 105. Each database computer can support connections from a multitude of bedside collection stations and the total number of servers required will depend on the total number of installed stations and the amount of network traffic on the system.

Data Collection Program

The data collected by the bedside units 101 and 110 and stored at 117 comprises vital signs/statistics, input and output quantities, service and pharmaceutical orders, and written and oral notes relating to the patient's overall status and is entered by the user at the patient's bedside. This data is collected at each patient's bedside using the browser-based data collection unit 101 which receives and displays HTML pages from the database server 105.

Before data collection begins, the bedside data system 102 first delivers a “login” screen that requires the operator to identify himself or herself by either manually entering an employee ID number or by employing the barcode scanner to enter a barcode from the user's identification card or badge. The submitted user's ID is then validated based on the employee records in the personnel database 123 maintained by the HIS 109. The entry of a password may be required for additional security against unauthorized use.

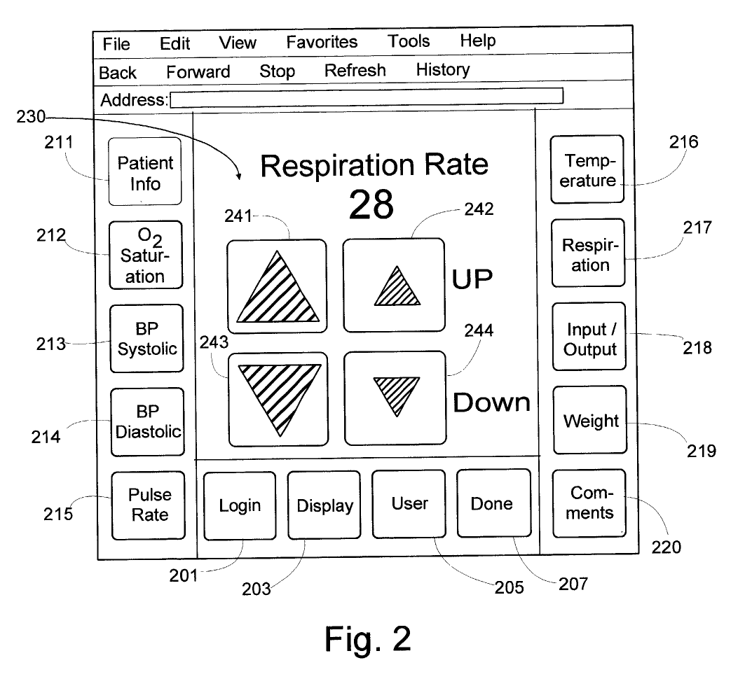

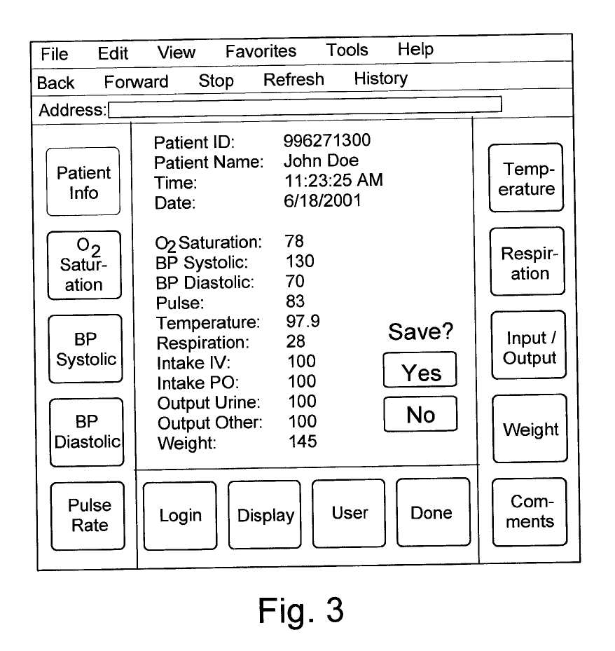

After an authorized operator has successfully completed the login operation, a data entry web page having the general layout depicted in FIG. 2 on the bedside unit is displayed. The data collection screen includes a set of navigation buttons at the bottom which select the system functions “Login” at 201 (permitting a new operator to log into the system as discussed above), “Display” at 203 which permits the operator to display data about the patient in different formats as described below, “Orders” at 205 which permits the entry of orders as described below, and “Done” at 207 which is pressed when all data has been collected with respect to a particular patient and which produces a confirmation display illustrated in FIG. 3 showing the data values entered for that patient to permit the operator to check the accuracy of the entries made.

By pressing the “Orders” button at 205, the operator can access display screens (not shown) which permit the entry of orders for tests, services, and/or medications for the patient in question at the bedside. In most cases, this feature will be a direct integration of existing HIS order-entry functions, but the bedside data system also provides its own order entry application for use in those cases where the HIS system lacks this feature.

At the beginning of each session, the bedside data system requires the identification of the patient whose data is to be collected. Again, the patient's name or identification code can be entered manually, or by scanning a barcode on the patient's wrist bracelet. The entered name or patient ID code is then validated against patient information in the HIS and a brief description of the patient will be displayed on the screen for the operator to verify and ensure that the ID was correct. The patient identification can be entered or altered by pressing the “Patient ID” button seen at 211 in FIG. 2.

Once both the operator and the patient have been correctly identified, the operator can enter any pertinent clinical data. The displayed “buttons” on the left and right sides of the data entry screen as seen in FIG. 2 permit the operator to select any of the following quantities for entry:

1. Oxygen saturation [212]

2. Blood pressure (systolic and diastolic) [213 and 214]

3. Pulse rate [215]

4. Temperature [216]

5. Respiration rate [217]

6. Intake/output (IV, PO, Urine, Other) [218]

7. Weight [219]

For each of the clinical quantities listed above, the operator will be presented with a different screen within the central frame area shown generally at 230. In FIG. 2, the central frame area 230 contains the pushbutton controls used to increment the current value for “Respiration Rate” which are displayed in response to pressing the button 217. Pressing any of the navigation buttons 212-219 displays central frame which displays the current setting for the selected quantity, and enables the operator to increment the displayed quantity upwardly or downwardly. In each case, a four-arrow button pattern consisting of a large-upward button 241, a small upward button 242, a large-downward button 243 and a small downward button 244, are displayed in the central frame 230. These buttons permit the operator to easily increment or decrement of the displayed value by a predefined large or small amount. Each time one of the buttons 241-244 is pressed, the bedside data system responds by sending a new web page to the bedside unit's browser program, thereby displaying the newly incremented value. The bedside data system presents the last entered value for the current patient as the starting value for each quantity (or displays a nominal default value for new patients having no prior history in the system). Each quantity 212-219 can be selected independently and none are required. This feature accommodates the specialized data collection needs of each patient.

By pressing the “Comments” button at 220, the operator can also enter various free form comments regarding the patient and/or the current data collection session. The comments may be manually entered using a displayed, on-screen keyboard (not shown) on a handheld unit, a conventional keyboard on a conventional PC, or dictated into an attached (optional) microphone for storage as a recording in the form of an MP3 file or the like. The recorded comments may be processed by speech recognition techniques to provide a draft transcript which may then be verified, and edited if need by a human to insure its accuracy, and stored as text data with the other bedside data at 117. The same microphone (not shown) may also be used to accept spoken commands and data values from the user, permitting hands-free entry of data values. When voice commands are used, the data values may be dictated in response to prompting messages displayed on the LCD screen, or in response to spoken prompts. A small loudspeaker (not shown) can be used to reproduce spoken prompts and previously recorded comment messages from the bedside terminal.

Session Summary

When the user has completed a data collection session for a particular patient and presses the “Done” button 207 seen in FIG. 2, the ‘Data Collection Summary’ screen is displayed (see FIG. 3). The summary screen lists each possible clinical quantity and its corresponding value for the current session. The user is given the opportunity to verify that the data was entered correctly, and to re-enter or modify erroneous items. Once the verification is complete, the user can specify whether the data is to be saved or discarded, and whether or not new collection session is to be started. If at any time the user fails to touch the screen or otherwise enter any data within a predetermined timeout period, the session will be erased and any entered data, including patient information, will be discarded.

The Analysis Screens

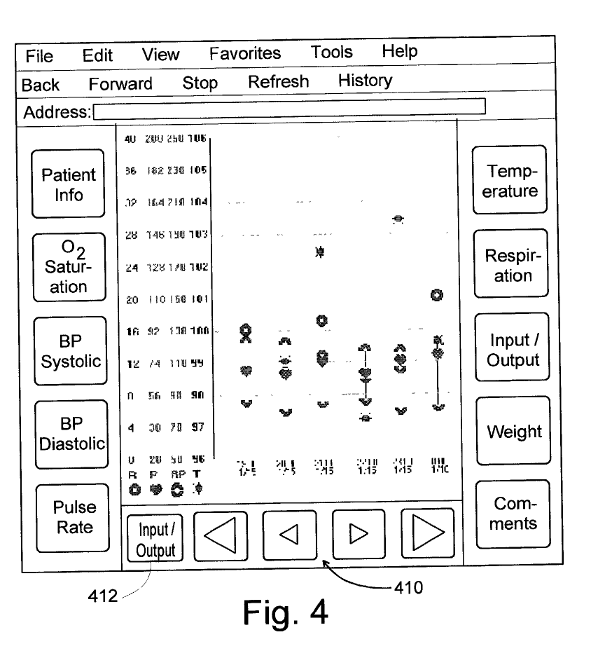

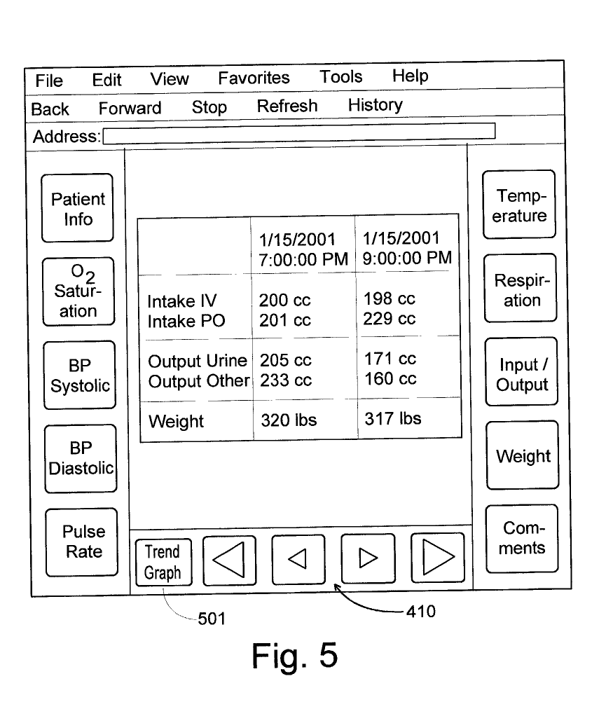

The operator may also press the “Display” button seen at 203 in FIG. 2 to view a display of the accumulated data for the currently selected patient as stored in the database 117 seen in FIG. 2. The display option contains two screens: the trend graph illustrated in FIG. 4 and the I/O table illustrated in FIG. 5.

The trend graph illustrated in FIG. 4 displays icons representing the patient's temperature, heart rate, respiration rate, and oxygen saturation on a multi-scaled graph. The user can opt to enable or disable point connections and trend lines. Using the familiar four-button group seen at 410, the operator can “scroll” the graph backward and forward in time by a single reading or by an entire shift. The value of each quantity is displayed by a small icon cursor on the graph using a heart-shaped icon to indicate heart rate, two concentric circles for respiration, a radiating star for temperature, an upwardly curved arc for systolic BP (blood pressure) and a downwardly curved arc for diastolic BP. In addition, when a color display or printout is used, each data symbol may be color coded as well as individually form-coded (using an icon) for ease of discrimination when viewing. The value of the data is shown as an auto-scale range for each quantity to the right of the graphical display.

By pressing the button marked “Input/Output” seen at 412, the operator may view an input/output table as illustrated in FIG. 5 for the same time period as selected on the graph of FIG. 4. All intakes and outputs are shown on the table, with corresponding patient weight readings and totals. Again, a four-button group as seen at 410 allows the user to scroll the table through time using the same increments as on the graph, and pressing button marked “Trend Graph” at 414 restores the trend view of FIG. 4.

The bedside data system is also capable of producing printed reports which include both trend graphs and tabular input/output data. These reports may then be made part of the documentation file for the patient. The clarity and accuracy of both the computer displays and the printed patient reports provide a significant improvement over the frequently illegible entries on the handwritten bedside charts commonly produced by conventional practices.

The printout (not shown) is composed of three sections. The first section is the graphical presentation of the patient vital signs (systolic BP, diastolic BP, heart rate, temperature and respiration rate) at each reporting interval. The report generation program also allows the selection and display of each of the quantities individually or in any combination. The value of each quantity is displayed by a small icon cursor on the graph using a heart-shaped icon to indicate heart rate, two concentric circles for respiration, a radiating star for temperature, an upwardly curved arc for systolic BP and a downwardly curved arc for diastolic BP. In addition, each data symbol is color coded as well as individually form-coded (using an icon) for ease of discrimination when viewing. The value of the data is shown as an auto-scale range for each quantity to the right of the graphical display.

Conclusion

It is to be understood that numerous modifications may be made to the specific embodiment that has been described without departing from the true spirit and scope of the invention.

What is claimed is:

1. The method of gathering, processing and displaying data describing a patient which comprises, in combination, the steps of:
employing a computer located at the bedside of said patient for accepting identification values which identify said patient and said operator,
employing said computer located at the bedside of said patient for accepting vital sign values from an operator which indicate the patient's current temperature, blood pressure, and pulse rate,
using a communications facility to transmit said vital sign values and said identification values to a remote computer from said computer located at the bedside of said patient to a remote computer,
storing said vital sign values and said identification values in a database,
employing said computer located at the bedside of said patient for transmitting a request for specified data previously stored in said database and for displaying data returned from said database in response to said request.

2. The method set forth in claim 1 wherein said computer located at the bedside of said patient includes a touchscreen for accepting said vital sign values from said operator.

3. The method set forth in claim 1 wherein said computer located at the bedside of said patent includes a touchscreen for displaying prompting messages which request specific ones of said vital sign values and for accepting said vital sign values from said operator.

4. The method set forth in claim 1 wherein computer located at said bedside includes a connected barcode scanner for accepting said identification values from a printed medium.

5. The method set forth in claim 2 wherein computer located at said bedside includes a connected barcode scanner for accepting said identification values from a printed medium.

6. The method set forth in claim 3 wherein computer located at said bedside includes a connected barcode scanner for accepting said identification values from a printed medium.

7. The method set forth in claim 1 wherein said computer located at said bedside comprises a handheld computer and said communications facility includes a radio communications link which transmits data between said handheld computer and said remote computer.

8. The method set forth in claim 1 wherein said computer located at said bedside comprises a handheld computer and said communications facility includes a radio communications link which transmits data between said handheld computer and said remote computer.

9. The method set forth in claim 3 wherein said computer located at said bedside comprises a handheld computer and said communications facility includes a radio communications link which transmits data between said handheld computer and said remote computer.

10. The method set forth in claim 9 wherein said handheld computer located at said bedside employs a barcode scanner for accepting said identification values from a printed medium.

11. The method set forth in claim 3 wherein said touch screen display a plurality of touch controls each of which increments or decrements the current value of said specific ones of said vital sign values.

12. In combination with a database server computer for storing patient data, a bedside computer for gathering, processing and displaying data describing a patient at the bedside of said patient and remote from said database server, said bedside computer patient comprising:
means for accepting identification values that identify said patient and an operator,
means for accepting vital sign values from said operator which indicate the patient's current temperature, blood pressure, and pulse rate,
a communications interface for transmitting said vital sign values and said identification values to said database computer for storage and for transmitting a request for specified data previously stored bu said database computer, and
means for displaying data returned from said database computer in response to said request.

13. The bedside computer set forth in claim 12 wherein said means for accepting vital sign values includes a touchscreen for accepting input commands from said operator.

14. The bedside computer set forth in claim 12 wherein said means for accepting vital sign values includes a touchscreen for displaying prompting messages which request specific ones of said vital sign values and for accepting said vital sign values from said operator.

15. The bedside computer set forth in claim 12 wherein said means for accepting identification values that identify said patient and an operator include a connected barcode scanner for accepting said identification values from a printed medium.

16. The bedside computer set forth in claim 13 wherein said means for accepting identification values that identify said patient and an operator include a connected barcode scanner for accepting said identification values from a printed medium.

15. The bedside computer set forth in claim 14 wherein said means for accepting identification values that identify said patient and an operator include a connected barcode scanner for accepting said identification values from a printed medium.

16. The method set forth in claim 12 wherein said bedside computer comprises a handheld computer and said communications facility includes a radio communications link which transmits data between said handheld computer and said remote computer.

17. The method set forth in claim 15 wherein said bedside computer comprises a handheld computer and said communications facility includes a radio communications link which transmits data between said handheld computer and said remote computer.
